(19) United States
(12) Patent Application Publication
(10) Pub. No.: US 2007/0051643 A1
(43) Pub. Date: Mar. 8, 2007

(54) Apparatus, system and method for storing an orthodontic appliance

(76) Inventor: Earl O. Bergersen, Dorado, PR (US)

Correspondence Address: PATENTS+TMS, P.C., 2849 W. ARMITAGE AVE., CHICAGO, IL, 60647, US

(21) Appl. No.: 11/514,668

(22) Filed: Sep. 1, 2006

Related U.S. Application Data
(60) Provisional application No. 60/715,351, filed on Sep. 8, 2005.

(51) Int. Cl. A61B 19/02; B65D 73/00

(52) U.S. Cl. 206/63.5; 206/368; 206/457

(57) ABSTRACT

An apparatus, a system and a method store an orthodontic appliance. The apparatus, the system and the method have a cover attachable to a base via a hinge. The base and/or the cover have exterior surfaces. The hinge may be in a closed position and/or an end of the base abuts an end of the cover. Interior surfaces of the base and/or the cover form, couple and/or combine to form an interior and/or a compartment for storing the orthodontic appliance. The interior and/or the compartment is sized to store, to receive, to surround and/or to house the orthodontic appliance. The exterior surfaces of the base and/or the cover couple, mate and/or join to form a shape resembling an object which may be visually recognizable to the user. A sticker, a decal, information, indicia and/or writing are applied to and/or are attached to the exterior surfaces of the base and/or of the cover for customization and/or for decoration by the user. A loop is attached to the base and/or to the cover for receiving a cord. The base, the cover and/or the orthodontic appliance may be worn by the user via the loop and/or the cord. Moreover, the base and/or the cover may be made from, for example, a light emitting material for locating the base and/or the cover in a dark environment.

This application claims the benefit of U.S. Provisional Application Ser. No.: 60/715,351, filed Sep. 8, 2005.

### BACKGROUND OF THE INVENTION

The present invention generally relates to an apparatus, a system and a method for storing an orthodontic appliance. More specifically, the present invention relates to an apparatus, a system and a method for storing an orthodontic appliance which may have a base attached to a cover by a hinge. The base and/or the cover may be sized to receive, to store, to house and/or to surround the orthodontic appliance. The base and/or the cover may have a tab or a lever to open and/or to close the hinge for separating the cover from the base. The base and/or the cover may have a loop for attaching a wire, a string and/or a cable to the base and/or the cover. A user may wear the base and/or the cover around a part of the body of the user via the string and/or the loop. With the hinge in a closed position, exterior surfaces of the base and of the cover may mate, may join and/or may couple to form a shape of an object, such as, a head of an animal, a vehicle, a mouth of a human and/or the like. A sticker, a decal, writing and/or indicia may be attached to and/or may be applied to the exterior surfaces of the base and/or the cover for the user to customize and/or to decorate the base and/or the cover. The sticker, the decal, the writing and/or the indicia may identify the user, an orthodontic technique associated with the orthodontic appliance, a manufacturer of the orthodontic appliance and/or a medical professional associated with the orthodontic appliance. The base, the cover and/or the loop may be made from a material, such as, for example, a light emitting material for locating the base and/or the cover in a dark environment.

It is generally known that a user may store an orthodontic appliance in a box to prevent damage to, to prevent destruction of, to prevent loss of and/or to prevent contamination of the orthodontic appliance. Typically, the box is formed in a shape, such as, for example, a square or a semicircle. Additionally, the box is larger than the orthodontic appliance to enable the orthodontic appliance to be stored inside the box and/or to be surrounded by the box. The user removes the orthodontic appliance from a mouth and/or a dentition of the user and may place and/or may store the orthodontic appliance in the box until the orthodontic appliance is subsequently used by the user. The user places the box which may store the orthodontic appliance in, for example, a pocket, a purse, a bag, a backpack and/or the like. The box has a bottom portion be connected to a top portion. A hinge attaches the bottom portion to the top portion. The box is accessed by separating the top portion from the bottom portion.

Typically, an exterior surface of the box has an appearance which is un-decorative and/or is uniform or plain. The user of the orthodontic appliance is often, for example a female, a male, a child, an adolescent or an adult. The appearance of the box may be unappealing to and/or uninteresting to the user. As a result, the user may be disinterested in using, in displaying and/or in wearing the orthodontic appliance because of the appearance of the box. Further, the appearance of the box may cause the user to not use and/or to not wear the orthodontic appliance as recommended and/or as instructed by a medical professional associated with the orthodontic appliance. Still further, the exterior surface of the box may not receive and/or may not display indicia and/or information relating to the user of the orthodontic appliance, to the orthodontic technique associated with the orthodontic appliance, to the manufacturer of the orthodontic appliance and/or to the medical professional. Moreover, the lack of interest in the box and/or the lack of appeal of the box to the user may result in the user losing, misplacing and/or damaging the box and/or the orthodontic appliance.

A need, therefore, exists for an apparatus, a system and a method for storing an orthodontic appliance. Additionally, a need exists for an apparatus, a system and a method for storing an orthodontic appliance which may be formed in a shape of an object, such as, a vehicle, an animal or a musical instrument. Further, a need exists for an apparatus, a system and a method for storing an orthodontic appliance which may be formed into a shape of and/or resembling an object which may be recognizable and/or may be visually appealing to a user, such as, a female, a male, a child, an adolescent and/or an adult. Still further, a need exists for an apparatus, a system and a method for storing an orthodontic appliance which may have a loop and/or a string for wearing the apparatus around a part of a body of a user. Moreover, a need exists for an apparatus, a system and a method for storing an orthodontic appliance which may receive and/or may display a sticker, a decal, information and/or indicia for customizing and/or for decorating the apparatus by a user. Furthermore, a need exists for an apparatus, a system and a method for storing an orthodontic appliance which may display indicia and/or information relating to a user, an orthodontic technique associated with the orthodontic appliance, a manufacturer of the orthodontic appliance and/or a medical professional associated with the orthodontic appliance.

### SUMMARY OF THE INVENTION

The present invention relates to an apparatus, a system and a method for storing an orthodontic appliance. The apparatus, the system and the method may have a first end of a cover attachable to a first end of a base via a hinge. The base and/or the cover may have an interior surface and/or an exterior surface. The hinge may be in a closed position and/or a second end of the base may abut a second end of the cover. The interior surfaces of the base and/or the cover may form, may couple and may combine to form an interior and/or a compartment for storing the orthodontic appliance. The interior and/or the compartment may be sized to store, to receive, to surround and/or to house the orthodontic appliance. The exterior surface of the base and/or the cover may couple, may mate and/or may join to form a shape resembling an object which may be recognizable and/or may be visually appealing to the user, such as, for example, a vehicle, a doll, a character, athletic equipment, an animal, a mouth of a human and/or the like. A sticker, a decal, indicia and/or writing may be applied to and/or may be attached to the exterior surface of the base and/or of the cover for customization and/or for decoration by the user. A loop may be attached to the base and/or to the cover for receiving a string, a wire, a cord and/or the like. The base, the cover and/or the orthodontic appliance may be worn by the user via the loop and/or the wire, the string and/or the cord. Moreover, the base and/or the cover may be made from, for example, a light emitting material for locating the base, the cover and/or the orthodontic appliance in a dark environment.

To this end, in an embodiment of the present invention, an apparatus for storing an orthodontic appliance is provided. The apparatus has a base having a length defined between a first end and a second end wherein the base has a first exterior surface defining a first interior. The base has a top side and a bottom side wherein the bottom side is opposite to the top side of the base wherein the first exterior surface of the base resembles a first portion of an object. Further, the apparatus has a cover having a length defined between a first end and a second end wherein the cover has a second exterior surface defining a second interior. The cover has a top side and a bottom side wherein the bottom side is opposite to the top side of the cover wherein the second exterior surface of the cover resembles a second portion of the object. Moreover, the apparatus has a hinge connecting the first end of the base and the first end of the cover wherein the cover moves between an open position and a closed position with respect to the base via the hinge wherein the bottom side of the base abuts the bottom side of the cover in the closed position. The first interior of the base and the second interior of the cover define a compartment between the base and the cover wherein the compartment is sized to receive the orthodontic appliance. The first exterior surface of the base aligns with the second exterior of the cover wherein a shape of the object is formed by the first portion of the object and the second portion of the object wherein the object is decorative.

In an embodiment, the apparatus has a loop connected to the first end of the cover wherein the loop extends outwardly with respect to the second exterior surface of the cover wherein the loop forms an opening between the second exterior surface of the cover and the loop.

In an embodiment, the apparatus has a cord connected the second exterior surface of the cover at the first end of the cover wherein the cord is made from any one of a fabric, a cloth, a plastic, a rubber, a polyurethane or a nylon.

In an embodiment, the apparatus has a tab connected to the second exterior surface of the cover wherein the tab extends outwardly with respect to the second end of the cover.

In an embodiment, the base is made from a light emitting material.

In an embodiment, the object resembles any one of an animal, a human being, a vehicle or a musical instrument.

In another embodiment of the present invention, a system for storing an orthodontic appliance of a user is provided. The system has a base having a length defined between a first end and a second end wherein the base has a first exterior surface defining a first interior wherein the base has a height defined between a top side and a bottom side. Further, the system has a cover having a length defined between a first end and a second end wherein the base has a second exterior surface defining a second interior wherein the cover has a height defined between a top side and a bottom side. Still further, the system has a hinge connected to the first end of the base and the first end of the cover wherein the cover moves between an open position and a closed position with respect to the base via the hinge. The bottom side of the base abuts the bottom side of the cover when the cover is located in the closed position wherein the first interior of the base and the second interior of the cover are sized to receive the orthodontic appliance. The first interior of the base and the second interior of the cover are enclosed by the first exterior surface of the base and the second exterior surface of the cover when the hinge is located in the first position. Moreover, the system has a cord connected to the second exterior surface of the cover wherein the cord is continuous and sized to be worn by the user.

In an embodiment, the system has a loop connected to the first end of the cover wherein the loop forms an opening between the first exterior surface of the cover and the loop.

In an embodiment, the system has a tab connected to the second end of the cover wherein the tab extends outwardly with respect to the second exterior surface of the cover.

In an embodiment, the cover is made from a light emitting material.

In an embodiment, the system has a decal connected to the base wherein the decal adheres to the first exterior surface of the base.

In an embodiment, the system has indicia displayed on the base wherein the indicia is adjacent to the first exterior surface of the base.

In an embodiment, a system has method for storing an orthodontic appliance is provided. The method has the step of providing a compartment having a base and a cover wherein the base has a first exterior surface and a length defined between a first end and a second end wherein the cover has a second exterior surface and a length defined between a first end and a second end. The first end of the base is connected to the first end of the cover wherein the cover moves between an open position and a closed position with respect to the base of the compartment wherein the second end of the cover contacts the second end of the base when the cover is located in the closed position. The first exterior surface of the base and the second exterior surface of the cover define an interior of the compartment wherein the interior of the compartment is sized to receive the orthodontic appliance. Further, the method has the step of forming a first portion of a shape on the first exterior surface of the base. Still further, the method has the step of forming a second portion of the shape on the second exterior surface of the cover wherein the cover moves to the closed position and the shape is formed by the first portion of the shape on the base and the second portion of the shape on the cover wherein the shape resembles an object which decorates the compartment.

In an embodiment, the method has the step of connecting a hinge between the first end of the base and the first end of the cover wherein the cover moves between the open position and the closed position via the hinge.

In an embodiment, the compartment is made from a light emitting material.

In an embodiment, the object resembles any one of an animal, a human being, a vehicle or a musical instrument.

In an embodiment, the method has the step of providing a decal associated with the object wherein the decal is attachable to the first exterior surface of the base.

In an embodiment, the method has the step of connecting a loop to the cover of the compartment wherein the loop forms a hole between the second exterior surface of the cover and the loop.

In an embodiment, the method has the step of connecting a tab to the second end of the cover wherein the tab extends outwardly with respect to the second exterior surface of the cover.

In an embodiment, the method has the step of displaying indicia on the compartment wherein the indicia is indicative of the orthodontic appliance.

It is, therefore, an advantage of the present invention to provide an apparatus, a system and a method for storing an orthodontic appliance which may be formed into a shape of an object visually appealing and/or recognizable to a user.

Another advantage of the present invention is to provide an apparatus, a system and a method for storing an orthodontic appliance which may resemble an object, such as, for example, an animal, a vehicle, an instrument, a mouth of a human.

And, another advantage of the present invention is to provide an apparatus, a system and a method for storing an orthodontic appliance which may have an interior or a compartment for storing the orthodontic appliance and/or an exterior surface formed in a shape resembling an object.

Yet another advantage of the present invention is to provide an apparatus, a system and a method for storing an orthodontic appliance which may a loop for attaching to a string, a wire, a cord and/or the like.

A further advantage of the present invention is to provide an apparatus, a system and a method for storing an orthodontic appliance which may be worn around a part of a body of a user.

Moreover, an advantage of the present invention is to provide an apparatus, a system and a method for storing an orthodontic appliance which may have a sticker, indicia, a decal and/or writing for customizing and/or for decorating the apparatus by a user and/or a medical professional associated with the orthodontic appliance.

And, another advantage of the present invention is to provide an apparatus, a system and a method for storing an orthodontic appliance which may receive and/or may display indicia and/or information relating to a user, a orthodontic technique associated with the orthodontic appliance, a manufacturer of the orthodontic appliance and/or a medical professional associated with the orthodontic appliance.

Yet another advantage of the present invention is to provide an apparatus, a system and a method for storing an orthodontic appliance which may an area on an exterior surfaces of a base and/or of a cover for receiving and/or for displaying indicia and/or information relating to a user, a orthodontic technique associated with the orthodontic appliance, a manufacturer of the orthodontic appliance and/or a medical professional associated with the orthodontic appliance.

Another advantage of the present invention is to provide an apparatus, a system and a method for storing an orthodontic appliance which may be made from a material, such as, for example, a light emitting material and/or the like.

Yet another advantage of the present invention is to provide an apparatus, a system and a method for storing an orthodontic appliance which may prevent damage to, destruction of, theft of and/or misplacement of the orthodontic appliance.

A still further advantage of the present invention is to provide an apparatus, a system and a method for storing an orthodontic appliance which may display an advertisement, a logo and/or a trademark associated with an orthodontic technique associated with the orthodontic appliance, a manufacture of the orthodontic appliance and/or a medical professional associated with the orthodontic appliance.

Moreover, an advantage of the present invention is to provide an apparatus, a system and a method for storing an orthodontic appliance which may allow and/or may aid a user in locating the apparatus in a dark environment.

And, another advantage of the present invention is to provide an apparatus, a system and a method for storing an orthodontic appliance which may improve, may heighten and/or may increase interest in and/or appeal of the orthodontic appliance by the user based on a visual appearance of the apparatus.

Yet another advantage of the present invention is to provide an apparatus, a system and a method for storing an orthodontic appliance which may improve, may heighten and/or may increase a willingness and/or a motivation to wear, to display and/or to use the orthodontic appliance by the user.

Additional features and advantages of the present invention are described in, and will be apparent from, the detailed description of the presently preferred embodiments and from the drawings.

### BRIEF DESCRIPTION OF THE DRAWINGS

FIG. 1 is a side plan view of an apparatus for storing an orthodontic appliance in an embodiment of the present invention.

FIG. 2 is a side plan view of an apparatus for storing an orthodontic appliance in another embodiment of the present invention.

FIG. 3 is a side plan view of an apparatus for storing an orthodontic appliance in another embodiment of the present invention.

FIG. 4 is a perspective view of an apparatus for storing an orthodontic appliance in another embodiment of the present invention.

FIG. 5 is a front plan view of an apparatus for storing an orthodontic appliance in another embodiment of the present invention.

FIG. 6 is a side plan view of the apparatus in FIG. 5 in an embodiment of the present invention.

FIG. 7 is a bottom plan view of an apparatus for storing an orthodontic appliance in another embodiment of the present invention.

### DETAILED DESCRIPTION OF THE PREFERRED EMBODIMENTS

An apparatus, a system and a method for storing an orthodontic appliance may have a base and/or a cover which may form a shape of an object, such as, for example, a vehicle, a head of an animal, a character, athletic equipment, a toy, a doll, a musical instrument, a mouth of a human and/or the like. The base and/or the cover may have an interior surface which may form an interior or a compartment for storing, for surrounding and/or for receiving the orthodontic appliance. A hinge may attach a first end of the cover to a first end of the base. A loop may be attached to the first end of the base and/or the first end of the cover for receiving a string, a wire or a cable. The base and the cover may be worn by a user to store, to carry and/or to transport the orthodontic appliance. A sticker, a decal, indicia and/or writing may be received and/or may be displayed on the base and/or the cover to identify the user, a technique associated with the orthodontic appliance, a manufacturer of the orthodontic appliance, a medical professional associated with the orthodontic appliance and/or the like. The base, the cover and/or the loop may be made of a material, such as, for example, a light emitting material for locating the base and/or the cover in a dark environment.

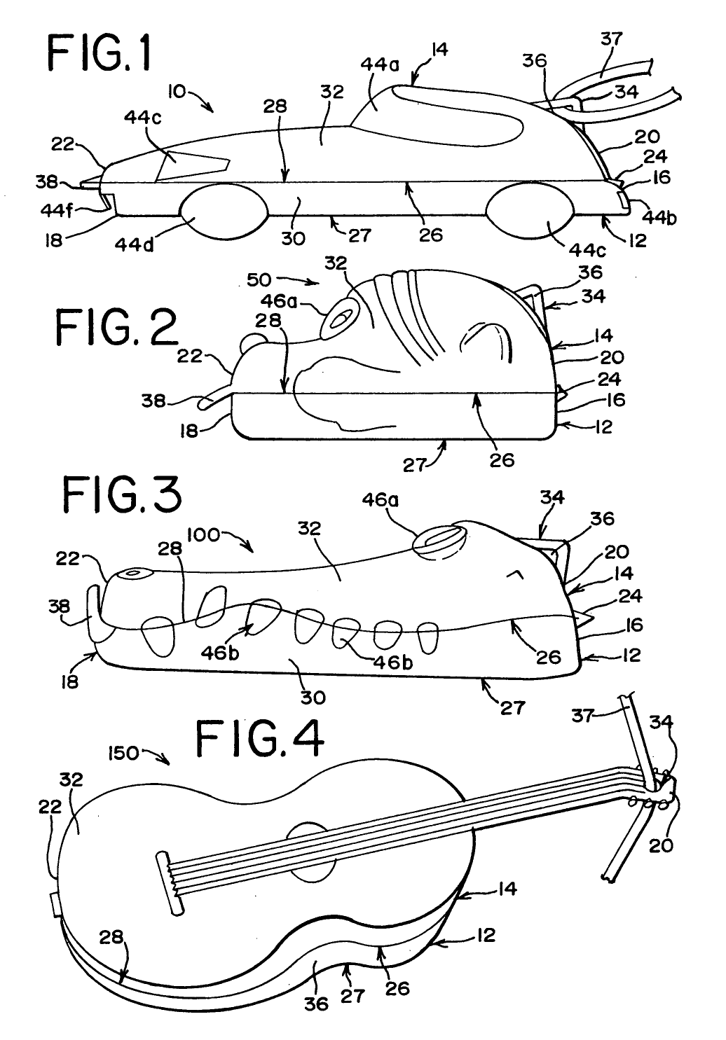

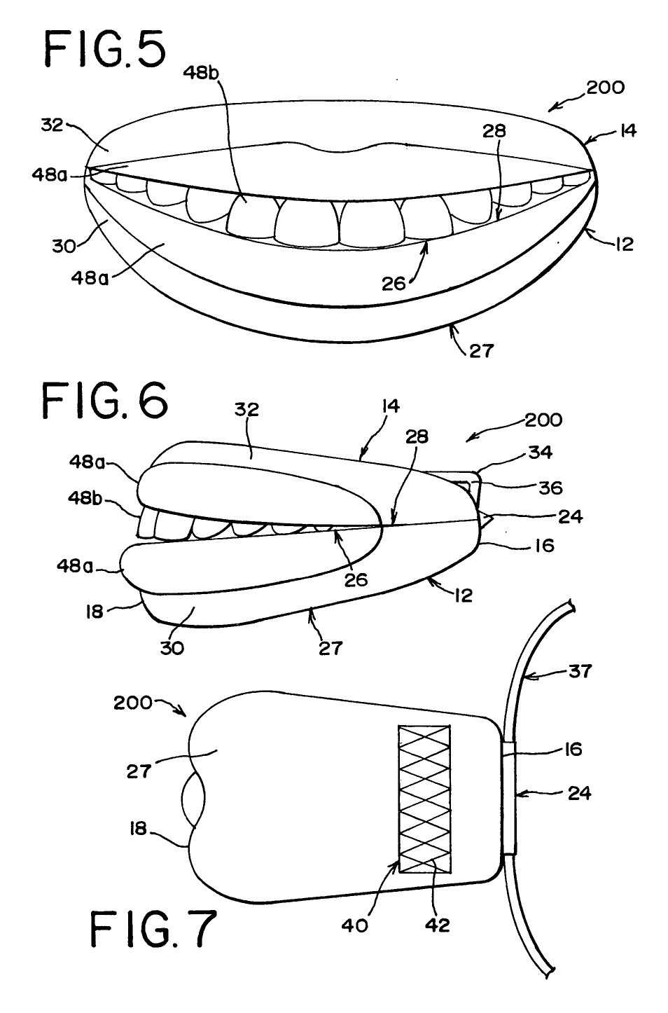

Referring now to the drawings wherein like numerals refer to like parts, FIGS. 1-4 and 6, illustrate a side plan view of apparatuses 10, 50, 100, 150, 200, respectively, for storing an orthodontic appliance (not shown in the figures) which may have a base 12 and/or a cover 14. The orthodontic appliance may be, for example, an orthodontic positioner, an orthodontic retainer and/or the like. The orthodontic appliance may be worn on a dentition of a mouth of a user (not shown in the figures). The user may be a male, a female, a child, an adolescent or an adult. The present invention should not be limited to a specific embodiment of the orthodontic appliance and/or the user. It should be understood that the orthodontic appliance may be any orthodontic appliance known to one having ordinary skill in the art.

The base 12 may have a first end 16 and/or a second end 18 which is opposite to the first end 16. The cover 14 may have a first end 20 and/or a second end 22 which is opposite to the first end 20. The base 12 may have a top side 26 and/or a bottom side 27 connecting the first end 16 to the second end 18. The top side 26 is opposite to the bottom side 27. The cover 14 may have a bottom side 28 connecting the first end 20 to the second end 22. The base 12 may have an interior (not shown in the drawings) and an exterior surface 30. The cover 14 may have an interior (not shown in the drawings) and an exterior surface 32.

A loop 34 may be connected to the exterior surface 32 of the cover 14 adjacent to the first end 20 of the cover 14. The loop 34 may extend outward with respect to the exterior surface 32 of the cover 14. A hole 36 may be located between the loop 34 and the exterior surface 32 of the cover 14. A cord 37 may be inserted into the hole 36 between the loop 34 and the cover 14. The cord 37 may be made from a material, such as, for example, a webbing, a fabric, a cloth, a plastic, a rubber, a polyurethane, a nylon, a string and/or the like. As a result, the cord 37 may be attached to the apparatuses 10, 50, 100, 150, 200 via the loop 34 and/or the hole 36. The user may wear the base 12 and/or the cover 14 around a part of the body via the cord 37 and/or the loop 34. As a result, the user may display, may transport and/or may carry the base 12, the cover 14 and/or the orthodontic appliance via the cord 37 and/or the loop 34. The part of the body of the user may be, for example, a neck, a wrist, a waist, an ankle and/or the like. The present invention should not be limited to a specific embodiment of the part of the body of the user and/or the material of the cord 37. It should be understood that the cord 37 may be made from any material as known to one of ordinary skill in the art.

A tab 38 may be connected to the exterior surface 32 of the cover 14 adjacent to the second end 22 of the cover 14 as shown in FIGS. 1, 2 and 4. The tab 38 may extend outward with respect to the exterior surface 32 of the cover 14. Alternatively, the tab 38 may be connected to the exterior surface 30 of the base 12 adjacent to the second end 18 of the base 12 as shown in FIG. 3. The tab 38 may extend outward with respect to the exterior surface 30 of the base 12.

A hinge 24 may attach the first end 16 of the base 12 to the first end 20 of the cover 14. The hinge 24 may have an open position and a closed position. The first end 20 of the cover 14 may be aligned with and/or may abut the first end 16 of the base 12. The tab 38 may move the hinge 24 between the open position and the closed position. In an open position of the hinge, the bottom side 28 of the cover 14 may be transverse with respect to the top side 26 of the base 12. As a result, the interiors of the base 12 and/or the cover 14 may be accessed, may be opened and/or may be exposed for receiving, for holding, for storing and/or for surrounding the orthodontic appliance. The hinge 24 may align the interior of the base 12 and/or the cover 14. The interior of the base 12 and/or the cover 14 may be sized to receive, to store and/or to house the orthodontic appliance. The interior of the base 12 and/or the cover 14 may be shaped to hold, to retain and/or to maintain the orthodontic appliance inside the interior of the base 12 and/or the cover 14. The interior of the base 12 and/or the cover 14 may have a shape corresponding to a shape of the orthodontic appliance to hold and/or to retain the orthodontic appliance inside the interior of the base 12 and/or the cover 14.

In a closed position of the hinge 24, the bottom side 28 of the cover 14 may abut and/or may be adjacent to the top side 26 of the base 12. Further, the second end 18 of the base 12 may be adjacent to and/or may abut the second end 22 of the cover 14. As a result, the interior of the base 12 and/or the cover 14 may be inaccessible, may be covered and/or may be protected by the exterior surfaces 30, 32 of the base 12 and/or the cover 14, respectively.

The orthodontic appliance may be placed in the interior of the base 12 and/or the cover 14 with the hinge 24 in the open position. The interior of the base 12 and/or the cover 14 may be sized to correspond the orthodontic appliance. In a preferred embodiment, the interior may have the dimensions between, for example, two inches by two inches by one inch or five inches by three and a half inches by three inches. The orthodontic appliance may be stored in, may be surrounded by, may be retained inside and/or may be enclosed in the interior of the base 12 and/or the cover 14 with the hinge 24 in the closed position. As a result, the orthodontic appliance may be stored in, may be housed in, may be retained inside, may be surrounded by and/or may be protected by the interior of the base 12 and/or the cover 14. The orthodontic appliance may be protected from damage, from destruction and/or from contamination by the exterior surfaces 30, 32 of the base 12 and/or the cover 14, respectively.

The base 12, the cover 14, the loop 34, the hinge 24 and/or the tab 38 may be formed by a technique, such as, for example, injection molding, compression molding and/or the like. The loop 34 and/or the tab 38 may be integrally formed with the base 12 and/or the cover 14 by the molding technique. The cover 14 and/or the hinge 24 may be integrally formed with the base 12 by the molding technique. The present invention should be not be limited to a specific embodiment of the molding technique of the base 12, the cover 14, the loop 34, the hinge 24 and/or the tab 38. It should be understood that the technique for forming the base 12, the cover 14, the loop 34, the hinge 24 and/or the tab 38 may be any technique know to one of ordinary skill in the art.

The base 12, the cover 14, the loop 34, the hinge 24 and/or the tab 38 may be made from a material, such as, for example, polyethylene, polypropylene, ABS, polycarbonate or polyurethane and/or the like. Alternatively, the material of the base 12, the cover 14, the loop 34, the hinge 24 and/or the tab 38 may be a “glow in the dark” material, such as, for example, a plastic resin having a photo-luminescent plastic resin, a zinc sulfide plastic resin or the like. The “glow in the dark” material may be visible by the user from a distance in a dark environment. As a result, the light emitting material may aid and/or may enable the user to locate the base 12, the cover 14 and/or the orthodontic appliance in the dark environment. The present invention should not be limited to a specific embodiment of the material and/or the “glow in the dark” material of the base 12, the cover 14, the loop 34, the hinge 24 and/or the tab 38.

As illustrated in FIG. 7, the apparatus 10, 50, 100, 150, 200 may have the bottom side 27 which may have an area 40 for receiving and for displaying indicia 42. The area 40 may have a roughened surface (not shown in the figures) and/or may be a course surface to receive and/or to display writing, ink, graphite, chalk and/or the like. The indicia 42 may be a number, a design, a logo, a trademark, an insignia, a word and/or a phrase of words which relate to the orthodontic appliance, a medical profession associated with the orthodontic appliance, an orthodontic technique associated the orthodontic appliance, an instruction for using the orthodontic technique and/or an entity, for example, a company manufacturing the orthodontic appliance and/or the base 12 and the cover 14. The indicia 42 may be information and/or may be writing which relates to the user, such as, for example, an identification of the user, a residence of the user, a telephone of the user, an address of the user and/or the like. The present invention should not be limited to a specific embodiment of the indicia 42, the information relating to the user and/or the entity associated with the orthodontic appliance.

The indicia 42 may be, for example, screened onto the area 42, and/or the exterior surfaces 30, 32 of the base 12 and/or the cover 14, respectively. The indicia 42 may be, for example, printed onto area 40 and/or the exterior surfaces 30, 32 of the base 12 and/or the cover 14, respectively. The base 12 and/or the cover 14 may be, for example, molded to contain the indicia 42. The indicia 42 may be contained on, for example, a front side of a sticker (not shown in the figures) having an adhesive backside which may be adhered to the area 40 and/or the exterior surfaces 30, 32 of the base 12 and/or the cover 14, respectively.

As illustrated in FIG. 1, the exterior surfaces 30, 32 of the base 12 and the cover 14 may be shaped as a vehicle, such as, for example, an automobile. The vehicle may be any vehicle, such as, for example, an airplane, a motorcycle, a truck, a boat and/or the like. The user may customize and/or may decorate the vehicle by attaching and/or affixing decals 44a-44f to the exterior surfaces 30, 32 of the base 12 and/or the cover 14. The image may be contained on, for example, a front side of the decals 44a-44f having an adhesive backside which may be adhered to the exterior surfaces 30, 32 of the base 12 and/or the cover 14, respectively. The image of the decals 44a-44f may correspond to an image of, for example, a windshield, a back license plate, a rear tire, a front tire, a head light and/or a front license plate, respectively. Furthermore, one or more stickers (not shown in the figures) may be attached to and/or may be affixed to the exterior surfaces 30, 32 of the base 12 and/or the cover 14 for customization and/or for decoration of the base 12 and/or the cover 14. The present invention should not be limited to a specific embodiment of the vehicle of the base 12 and/or the cover 14.

As illustrated in FIGS. 2 and 3, the exterior surfaces 30, 32 of the base 12 and the cover 14 may be shaped as a body part of an animal, such as, for example, a head of a monkey and/or a head of a crocodile, respectively. The head may be any animal, such as, for example, a dog, a cat, a lion, an eagle and/or the like. The user may customize and/or may decorate the head of the animal by attaching and/or affixing decals 46a, 46b to the exterior surfaces 30, 32 of the base 12 and/or the cover 14. The image may be contained on, for example, a front side of decals 46a, 46b having an adhesive backside which may be adhered to the exterior surfaces 30, 32 of the base 12 and/or the cover 14, respectively. The image of the decals 46a, 46b may correspond to an image of eyes and/or teeth, respectively, of the animal. The present invention should not be limited to a specific embodiment of the animal and/or the part of the animal.

As illustrated in FIG. 4, the exterior surfaces 30, 32 of the base 12 and the cover 14 may be shaped as a musical instrument, such as, for example, guitar. The musical instrument may be any musical instrument, such as, for example, a violin, a drum, a flute and/or the like. The user may customize and/or may decorate the musical instrument by attaching and/or by affixing decals (not shown in the drawings) to the exterior surfaces 30, 32 of the base 12 and/or the cover 14. The present invention should not be limited to a specific embodiment of the musical instrument.

As illustrated in FIGS. 5-7, the exterior surfaces 30, 32 of the base 12 and the cover 14 may be shaped as a body part of a human, such as, for example, a mouth and teeth of a human, respectively. The base 12 may resemble a bottom lip and a bottom dentition of the mouth of the human. The cover 14 may resemble a top lip and/or a top dentition of the mouth of the human. The bottom side 28 of the cover 14 may abut the top side 26 of the base 12 similarly to the top dentition abutting the bottom dentition of the mouth of the human. The user may customize and/or may decorate the mouth and the teeth of the human by attaching and/or affixing decals 48a, 48b to the exterior surfaces 30, 32 of the base 12 and/or the cover 14. The image may be contained on, for example, a front side of decals 48a, 48b having an adhesive backside which may be adhered to the exterior surfaces 30, 32 of the base 12 and/or the cover 14, respectively. The image of the decals 48a, 48b may correspond to images of lips and/or teeth, respectively, of a human. The present invention should not be limited to a specific embodiment of the body part of the human.

The exterior surfaces 30, 32 of the base 12 and the cover 14 may have a shape resembling an object which may be recognizable, may be visually appealing and/or may be visually interesting to the user. The tab 38 may be within a natural anatomy of the object of the base 12 and the cover 14. For example, the tab 38 may be the front license plate and/or a front bumper of vehicle, such as, for example, an automobile. The object may be, for example, the vehicle, the body part of the animal, the musical instrument, sports equipment, a toy, a doll and/or the like. The shape of the base 12 and the cover 14 may heighten and/or may increase an interest in and/or an appeal of the orthodontic appliance to the user. The interest and/or the appeal of the user caused by the shape of the base 12 and/or the cover 14 may increase use of the orthodontic appliance by the user. As a result, the user may wear and/or use the orthodontic appliance as instructed by the medical professional associated with the orthodontic appliance. It should be understood that the object may be any object which may be recognizable, may be visually appealing and/or may be visually interesting to the user.

A user may remove the orthodontic appliance from a mouth of the user and/or a dentition of the mouth of the user. The user may apply a force to the tab 38 for moving the hinge 24 from the closed position to the open position. The force applied by the user may be in an outward direction with respect to the second end 16 of the base 12. As a result, the hinge 24 may open, and the second end 22 and/or the bottom side 28 of the cover 14 may be separated from the second end 18 and/or the top side 26, respectively, of the base 12. The orthodontic appliance may be inserted into and/or may be received by the interior of the base 12 and/or the cover 14. As a result, the orthodontic appliance may be stored in, may be housed by and/or may be enclosed by the base 12 and/or the cover 14.

The interior of the base 12 and/or the cover 14 may be sized to retain, to hold and/or to maintain the orthodontic appliance inside the interior of the base 12 and/or the cover without application of an exterior force by the user. The interior of the base 12 and/or the cover 14 may have the shape corresponding to the shape of the orthodontic appliance. The shape of the interior of the base 12 and/or the cover 14 may hold, may retain and/or may maintain the orthodontic appliance inside the interiors of the base 12 and/or the cover 14. Moreover, the interior of the base 12 and/or the cover 14 may have a protrusion (not shown in the drawings) extending outward with respect to the interior of the base 12 and/or the cover 14. The protrusion may be, for example, a flange, a rib, a tab and/or the like. The protrusion may engage, may retain and/or may hold the orthodontic appliance inside the interior of the base 12 and/or the cover 14. As a result, the protrusion and/or the interior of the base 12 and/or the cover 14 may attach the orthodontic appliance to the base 12 and/or the cover 14.

The user may apply a force to the tab 38 and/or exterior surface 32 of the cover 30 to move and/or to close the hinge from the open position to the closed position. The bottom side 28 and/or the second end 22 of the cover 14 may abut the top side 26 and/or the second end 18 of the base 14. As a result, access to the interior of the base 12 and/or the cover 14 may be closed and/or may be prevented with the hinge 24 in the closed position. The base 12 and the cover 14 may store, may house and/or may protect the orthodontic appliance from damage, from destruction and/or from contamination with the hinge 24 in the closed position.

The user may wear the base 12, the cover 14 and/or the orthodontic appliance around the body part, such as, for example, the neck of the user via the loop 34 and/or the cord 37. The base 12, the cover 14 and/or the orthodontic appliance may be attached to the user via the loop 34 and/or the cord 37. As a result, the base 12, the cover 14, the loop 34 and/or the cord 37 may prevent the user from losing, from misplacing and/or from disregarding the orthodontic appliance. The user may wear the base 12, the cover 14 and/or the orthodontic appliance to carry, to transport and/or to move the orthodontic appliance. The user may remove the orthodontic appliance from the base 12 and/or the cover 14 to insert the orthodontic appliance into the mouth of the user. The user may wear the orthodontic appliance in the dentition of the mouth of the user.

The user may wear the base 12 and/or the cover 14 around the body part of the user. The indicia 42 on the base 12 and/or the base 14 may be displayed to and/or may be viewed by a customer. The customer may be a male, a female, a child, an adolescent or an adult. The indicia may advertise the entity, the orthodontic technique, the manufacturer, the user and/or the medical professional to the customer of the base 12 and/or the cover 14. As a result, the customer may be enticed, may be urged and/or may be influenced to purchase the orthodontic appliance and/or to contact the medical professional by the indicia 42 and/or the shape of the base 12 and/or the cover 14.

The apparatuses 10, 50, 100, 150, 200 may have the first end 20 of the cover 14 attachable to the first end 16 of the base 12 via the hinge 24. The base 12 and/or the cover 14 may have the interior surface and/or the exterior surfaces, 30, 32, respectively. The hinge 24 may be in the closed position and/or the second end 18 of the base 12 may abut the second end 22 of the cover 14. The interior surfaces of the base 12 and/or the cover 14 may form, may couple and may combine to form an interior and/or a compartment for storing the orthodontic appliance. The interior and/or the compartment may be sized to store, to receive, to surround, to retain and/or to house the orthodontic appliance. The exterior surfaces 30, 32 of the base 12 and/or the cover 14 may couple, may mate and/or may join to form a shape resembling an object which may be recognizable and/or may be visually appealing to the user. A sticker, a decal, indicia and/or writing may be applied to and/or may be attached to the exterior surfaces 30, 32 of the base 12 and/or of the cover 14, respectively for customization and/or for decoration by the user. A loop 34 may be attached to the base 12 and/or to the cover 14 for receiving a cord 37. The base 12, the cover 14 and/or the orthodontic appliance may be worn by the user via the loop 34 and/or the cord 37. Moreover, the base 12 and/or the cover 14 may be made from, for example, a light emitting material for locating the base 12 and/or the cover 14 in a dark environment.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

I claim:

1. An apparatus for storing an orthodontic appliance, the apparatus comprising:
a base having a length defined between a first end and a second end wherein the base has a first exterior surface defining a first interior wherein the base has a top side and a bottom side wherein the bottom side is opposite to the top side of the base wherein the first exterior surface of the base resembles a first portion of an object;
a cover having a length defined between a first end and a second end wherein the cover has a second exterior surface defining a second interior wherein the cover has a top side and a bottom side wherein the bottom side is opposite to the top side of the cover wherein the second exterior surface of the cover resembles a second portion of the object; and
a hinge connecting the first end of the base and the first end of the cover wherein the cover moves between an open position and a closed position with respect to the base via the hinge wherein the bottom side of the base abuts the bottom side of the cover in the closed position wherein the first interior of the base and the second interior of the cover define a compartment between the base and the cover wherein the compartment is sized to receive the orthodontic appliance wherein the first exterior surface of the base aligns with the second exterior of the cover wherein a shape of the object is formed by the first portion of the object and the second portion of the object wherein the object is decorative.

2. The apparatus of claim 1 further comprising:
a loop connected to the first end of the cover wherein the loop extends outwardly with respect to the second exterior surface of the cover wherein the loop forms an opening between the second exterior surface of the cover and the loop.

3. The apparatus of claim 1 further comprising:
a cord connected the second exterior surface of the cover at the first end of the cover wherein the cord is made from any one of a fabric, a cloth, a plastic, a rubber, a polyurethane or a nylon.

4. The apparatus of claim 1 further comprising:
a tab connected to the second exterior surface of the cover wherein the tab extends outwardly with respect to the second end of the cover.

5. The apparatus of claim 1 wherein the base is made from a light emitting material.

6. The apparatus of claim 1 wherein the object resembles any one of an animal, a human being, a vehicle or a musical instrument.

7. A system for storing an orthodontic appliance of a user, the system comprising:
a base having a length defined between a first end and a second end wherein the base has a first exterior surface defining a first interior wherein the base has a height defined between a top side and a bottom side;
a cover having a length defined between a first end and a second end wherein the base has a second exterior surface defining a second interior wherein the cover has a height defined between a top side and a bottom side;
a hinge connected to the first end of the base and the first end of the cover wherein the cover moves between an open position and a closed position with respect to the base via the hinge wherein the bottom side of the base abuts the bottom side of the cover when the cover is located in the closed position wherein the first interior of the base and the second interior of the cover are sized to receive the orthodontic appliance wherein the first interior of the base and the second interior of the cover are enclosed by the first exterior surface of the base and the second exterior surface of the cover when the hinge is located in the first position; and
a cord connected to the second exterior surface of the cover wherein the cord is continuous and sized to be worn by the user.

8. The system of claim 7 further comprising:
a loop connected to the first end of the cover wherein the loop forms an opening between the first exterior surface of the cover and the loop.

9. The system of claim 7 further comprising:
a tab connected to the second end of the cover wherein the tab extends outwardly with respect to the second exterior surface of the cover.

10. The system of claim 7 wherein the cover is made from a light emitting material.

11. The system of claim 7 further comprising:
a decal connected to the base wherein the decal adheres to the first exterior surface of the base.

12. The system of claim 7 further comprising:
indicia displayed on the base wherein the indicia is adjacent to the first exterior surface of the base.

13. A method for storing an orthodontic appliance, the method comprising the steps of:
providing a compartment having a base and a cover wherein the base has a first exterior surface and a length defined between a first end and a second end wherein the cover has a second exterior surface and a length defined between a first end and a second end wherein the first end of the base is connected to the first end of the cover wherein the cover moves between an open position and a closed position with respect to the base of the compartment wherein the second end of the cover contacts the second end of the base when the cover is located in the closed position wherein the first exterior surface of the base and the second exterior surface of the cover define an interior of the compartment wherein the interior of the compartment is sized to receive the orthodontic appliance;
forming a first portion of a shape on the first exterior surface of the base; and
forming a second portion of the shape on the second exterior surface of the cover wherein the cover moves to the closed position and the shape is formed by the first portion of the shape on the base and the second portion of the shape on the cover wherein the shape resembles an object which decorates the compartment.

14. The method of claim 13 further comprising the step of:
connecting a hinge between the first end of the base and the first end of the cover wherein the cover moves between the open position and the closed position via the hinge.

15. The method of claim 13 wherein the compartment is made from a light emitting material.

16. The method of claim 13 wherein the object resembles any one of an animal, a human being, a vehicle or a musical instrument.

17. The method of claim 13 further comprising the step of:
providing a decal associated with the object wherein the decal is attachable to the first exterior surface of the base.

18. The method of claim 13 further comprising the step of:
connecting a loop to the cover of the compartment wherein the loop forms a hole between the second exterior surface of the cover and the loop.

19. The method of claim 13 further comprising the step of:
connecting a tab to the second end of the cover wherein the tab extends outwardly with respect to the second exterior surface of the cover.

20. The method of claim 13 further comprising the step of:
displaying indicia on the compartment wherein the indicia is indicative of the orthodontic appliance.
